(19) United States
(12) Patent Application Publication
(10) Pub. No.: US 2025/0243129 A1
(43) Pub. Date: Jul. 31, 2025

(54) Ambient, Catalyst-Free Synthesis of Ammonia, Amino Acids, and Urea via Bubble-Induced Microenvironments

(71) Applicant: Jerome Cuomo, Cary, NC (US)

(72) Inventor: Jerome Cuomo, Cary, NC (US)

(21) Appl. No.: 19/183,880

(22) Filed: Apr. 20, 2025

Related U.S. Application Data
(60) Provisional application No. 63/676,624, filed on Jul. 29, 2024.

(51) Int. Cl. C05C 1/00; B01J 10/00; B01J 19/00; C01C 1/18; C05C 9/00; C05C 11/00; C05G 5/23; C07C 227/08; C07C 273/04

(52) U.S. Cl. CPC C05C 1/00; B01J 10/00; B01J 19/008; C01C 1/185; C05C 9/00; C05C 11/00; C05G 5/23; C07C 227/08; C07C 273/04

(57) ABSTRACT

A method and system are provided for synthesizing nitrogen-containing compounds, including ammonia, urea, amino acids, and ammonium salts, under ambient temperature and pressure conditions without the use of external catalysts or high-energy input. A nitrogen-containing gas and water are introduced into an aqueous solution through a submerged bubble-diffusing component, generating microbubbles that collapse and rupture to form high-energy microenvironments. These environments facilitate in situ molecular dissociation and the formation of reactive nitrogen species, including ammonia as an intermediate. The method supports reaction with co-solutes such as carbon dioxide, organic acids, or inorganic anions to yield target compounds. Optional low-energy augmentations—such as ultraviolet irradiation, ultrasonic agitation, or shear mixing—may enhance yield and selectivity. The system is compatible with isotopic gas variants and can be configured for batch or continuous-flow operation. Applications include decentralized production of fertilizers and biochemical precursors in agricultural, hydroponic, or laboratory settings.

### CROSS-REFERENCE TO RELATED APPLICATIONS

This application claims the benefit of U.S. Provisional Patent Application No. 63/676,624, filed Jul. 29, 2024, titled “Devices, Systems, and Methods for Molecular Conversion and Synthesis”; U.S. Provisional Patent Application No. 63/693,279, filed Sep. 11, 2024, titled “Devices, Systems, and Methods for Molecular Conversion and Synthesis”; and U.S. Provisional Patent Application No. 63/744,779, filed Jan. 13, 2025, titled “One-Step Ambient and Non-Catalytic Synthesis of Ammonia and Ammonium Compounds.” The contents of each of these provisional applications are incorporated herein by reference in their entirety.

### BACKGROUND OF THE INVENTION

### Field of the Invention

The present invention relates to the field of chemical synthesis and molecular transformation. More specifically, it pertains to methods and systems for nitrogen fixation and nitrogen-based compound synthesis in aqueous environments.

### Description of the Related Art

Nitrogen-based compounds—including ammonia, amino acids, and urea—are essential precursors in agriculture, pharmaceuticals, and chemical manufacturing. These compounds support global food security, medical formulation, and a wide range of industrial processes. Among them, ammonia plays a central role as both a product and intermediate, especially in fertilizer and urea synthesis. Historically, the production of these compounds at industrial scale has relied heavily on the Haber-Bosch process, which reacts molecular nitrogen (N₂) with hydrogen gas (H₂) to produce ammonia (NH₃) under elevated temperatures (400-500° C.) and high pressures (150-300 atm), typically in the presence of iron-based catalysts.

While the Haber-Bosch process was a breakthrough in early 20th-century chemistry, it remains one of the most energy- and carbon-intensive industrial processes in use today. Global ammonia production accounts for over 1.5% of total carbon dioxide emissions, driven primarily by the high energy requirements for hydrogen generation and the thermal conditions necessary for reaction equilibrium. With global demand for nitrogen-based compounds continuing to rise, there is an urgent need for alternative synthesis pathways that reduce energy input, lower emissions, and allow for decentralized or small-scale production.

A variety of alternatives have been proposed, including microbial fermentation, electrochemical nitrogen reduction, and heterogeneous catalysis using advanced materials such as ruthenium, iridium, or molybdenum. These methods, however, often depend on multi-stage systems, rare catalytic materials, or specialized equipment, and may require pressurized or high-temperature conditions. Furthermore, many of these approaches are limited in their ability to generalize across compound types (e.g., ammonia versus amino acids) and frequently involve downstream processing that constrains real-time or point-of-use applications.

In light of these challenges, there remains a gap for simple, low-energy, and catalyst-free methods capable of producing a range of nitrogen-based compounds under ambient conditions. Existing art has not demonstrated such methods using experimentally validated, scalable, and catalyst-independent systems.

### Comparison to Prior Art

Various strategies have been investigated to provide alternatives to the Haber-Bosch process for the synthesis of ammonia and related nitrogen-containing compounds. These include heterogeneous catalysis using advanced materials, theoretical models based on microbubble phenomena, and high-energy methods involving plasma-assisted chemistry. While these approaches offer conceptual advancements, several limitations in operational conditions, scalability, or reliance on specialized components remain.

Kitano et al. (2018), in Nature Communications, describe a ruthenium catalyst supported on a C12A7 electride substrate that promotes ammonia synthesis via reactive hydrogen species. The system is capable of operating at lower temperatures than Haber-Bosch but still requires elevated thermal conditions (˜400° C.), high-purity gas feeds, and relies on noble metal catalysts and electride materials, which may be cost-prohibitive or difficult to scale.

Kubicsek and Hegedüs (2025), in Periodica Polytechnica Chemical Engineering, present a computational study evaluating the energy intensity of ammonia production via freely oscillating microbubbles. Using a numerical model based on the Keller-Miksis equation, the authors analyze thermodynamic parameters and estimate the energetic efficiency of microbubble-induced nitrogen fixation. While the study quantifies the energy demand relative to conventional methods such as Haber-Bosch, it does not experimentally validate ammonia formation or demonstrate a complete synthesis pathway.

U.S. Pat. No. 9,272,920 B2 discloses a system for synthesizing ammonia using nano-sized metal and metal alloy catalyst particles. The approach leverages surface interactions at the nanoscale to enhance reaction efficiency and operates under externally applied energy input with controlled reactant streams. While the method offers improved kinetics relative to conventional catalysis, it depends on specialized catalytic materials and operating conditions that may increase complexity and cost in practical applications.

Wei et al. (2024), in Chemistry-A European Journal, investigate prebiotic peptide formation through bubbling combined with arc plasma discharge. While this method demonstrates that gas-liquid interactions may promote biomolecular synthesis, it requires external electrical input and high-energy plasma systems. The system is not directed at the synthesis of ammonia or urea. Furthermore, it operates under non-ambient conditions, relying on externally applied plasma fields.

These prior works demonstrate important progress in the field of nitrogen fixation and nitrogen compound synthesis. However, they often rely on catalysts, elevated temperatures, or theoretical assumptions, and are typically limited in scope to specific compounds or reaction environments. A need remains for experimentally validated, low-energy systems capable of producing a range of nitrogen-based compounds under standard environmental conditions without reliance on rare materials or external energy sources.

### SUMMARY OF THE INVENTION

The invention provides a one-step, ambient-temperature method for synthesizing ammonia and nitrogen-based compounds-including urea and amino acids-using only water and a nitrogen-containing gas such as air or molecular nitrogen (N₂). Under standard atmospheric conditions, gas is introduced into an aqueous solution through a bubble-diffusing element, such as a porous ceramic or stainless-steel bubbler. This generates microbubbles that collapse and rupture within the solution and at the gas-liquid interface, producing transient, high-energy microenvironments that drive chemical transformations. Notably, the process operates without the need for catalysts, elevated temperature or pressure, or externally applied energy sources.

Among the reactive species formed, atomic hydrogen is believed to play a central role in enabling nitrogen activation and compound formation. The downstream formation of urea, amino acid derivatives, and inorganic ammonium salts indicates that ammonia is generated in situ as an intermediate through the bubble-bursting process. Each of these products may serve as a key component in fertilizer or biochemical formulations.

The process can be modified or enhanced through optional additions to the gas or solution phase. Dissolved carbon dioxide may be introduced to promote urea formation, while simple carbon-containing acids such as acetic acid can support amino acid synthesis. Inorganic anions, including chloride and sulfate, may also be included to enable the formation of ammonium salts. The method is compatible with isotopically labeled variants, including nitrogen-15, carbon-13, and carbon-14, and can be adapted for use with enriched or alternative nitrogen-containing gas mixtures, depending on the application-such as isotopic tracing, diagnostic studies, or reaction pathway optimization.

Additional energy input may be applied to augment system performance through low-intensity enhancements including ultraviolet irradiation, ultrasonic vibration, or shear mixing. These techniques may increase cavitation intensity, improve yield, suitable for both large-scale and small-scale deployment scenarios, including batch and continuous-flow configurations.

The invention offers a practical and scalable approach to nitrogen compound synthesis in aqueous environments. Its ability to operate under ambient conditions and without specialized infrastructure enables on-site production of valuable compounds with minimal energy input or environmental impact. This makes it well suited for distributed or resource-limited settings.

Potential applications include localized fertilizer generation for agricultural and hydroponic systems, decentralized synthesis of precursors for biochemical manufacturing, and point-of-use production of nitrogen-based materials. The method's adaptability and operational simplicity make it suitable for both large-scale and small-scale deployment scenarios.

### BRIEF DESCRIPTION OF THE DRAWINGS

FIG. 1—Bubble-Based Reaction Setup: Diagram of the experimental setup showing a gas inlet, submerged porous bubbler, aqueous reaction medium, and optional energy inputs such as UV and ultrasound. This system enables ambient-condition synthesis through controlled microbubble generation and rupture within a liquid-phase environment.

FIG. 2—Bubble Dynamics and Microenvironment Generation: Conceptual illustration showing stages of bubble formation, rupture, and localized energy release. Highlights the role of microbubble collapse in generating transient high-energy zones, where molecular dissociation of nitrogen and water occurs, forming reactive intermediates essential to compound synthesis.

FIG. 3—Reaction Pathways Overview: Flowchart showing the transformation of nitrogen gas and water into reactive species, leading to ammonia formation. Ammonia then reacts with dissolved carbon dioxide or organic acids to produce urea and amino acids, respectively, all under ambient conditions and without a catalyst.

FIG. 4—Raman Spectra Comparison: Overlaid Raman spectra comparing synthesized product to a commercial urea standard. Key vibrational peaks near 977, 1311, 1556, and 2950 cm⁻¹ confirm the structural presence of urea and related nitrogen-containing compounds formed during the bubble-bursting process.

FIG. 5—Ninhydrin Test Result: Microscopic image of a ninhydrin-stained reaction sample. Shows distinct purple coloration associated with Ruhemann's purple, confirming the presence of free amine groups-strong evidence for the formation of amino acids under ambient reaction conditions.

FIG. 6—Microscopy Pattern Illustration: Composite figure comparing experimental microscopy images to known peptide-like structures. Includes 1000X optical images from the bubble-bursting system and reference models, showing morphological alignment and folding patterns consistent with early-stage peptide assembly and molecular self-organization.

### DETAILED DESCRIPTION OF THE INVENTION

### System Overview and Reaction Environment

In a preferred embodiment, the invention provides a one-step, catalyst-free process for synthesizing ammonia and nitrogen-containing compounds—for example, urea and amino acids—under ambient temperature and pressure conditions. The process requires only water and a nitrogen-containing gas, such as atmospheric air or molecular nitrogen (N₂).

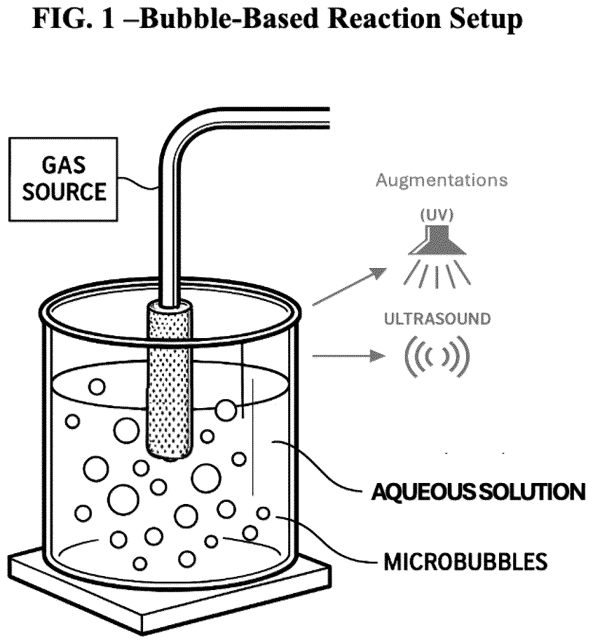

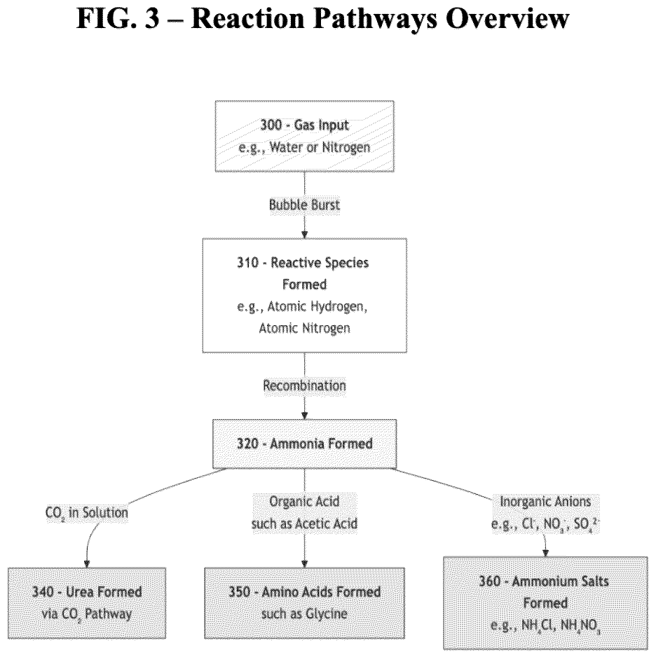

As illustrated in FIG. 3, the process begins with the introduction of a gas and liquid feed (element 300) into a reaction system designed to support continuous gas-liquid interaction. As shown in FIG. 1, the system includes a gas supply connected to a submerged bubble-diffusing component—such as a porous ceramic or stainless-steel stone bubbler—configured to generate microbubbles within an aqueous medium. These microbubbles propagate throughout the liquid, establishing the reactive interface required for subsequent molecular transformations.

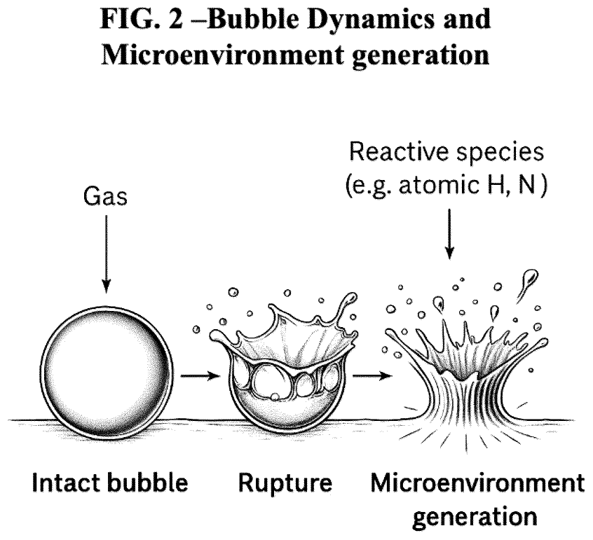

Once introduced, the bubbles undergo cycles of expansion and collapse within the liquid medium and ruptures upon reaching the gas-liquid interface. These dynamic events generate transient, localized high-energy microenvironments sufficient to initiate molecular dissociation. The resulting conditions facilitate the formation of reactive species, such as atomic hydrogen and atomic nitrogen (FIG. 3, element 310). The energy dynamics associated with bubble collapse and the spatial zones where reactivity is most pronounced are conceptually represented in FIG. 2, which outlines the progression of bubble evolution and likely sites of chemical activation.

### Empirical Evidence of Product Formation

Experimental results support the in situ generation of ammonia and its conversion into a variety of nitrogen-based compounds. These findings are confirmed by multiple analytical techniques, including colorimetric testing, spectroscopy, and microscopy.

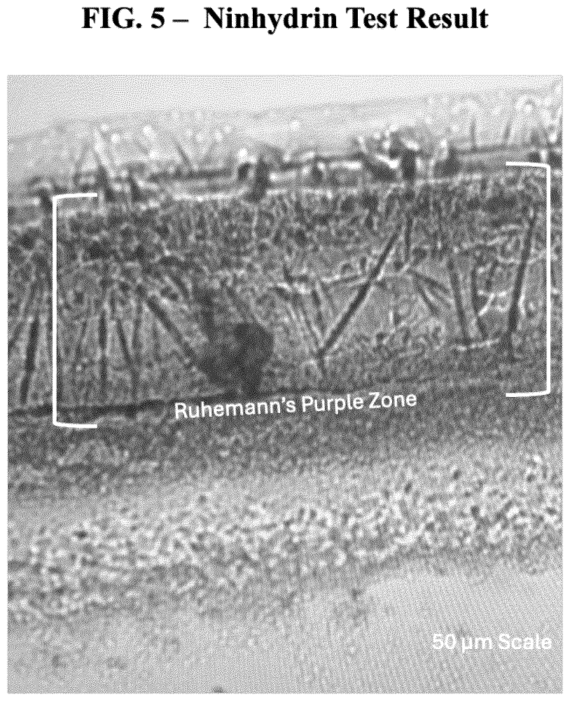

Colorimetric analysis using ninhydrin produced the deep purple chromophore known as Ruhemann's purple, a widely accepted indicator of free amine groups. Given that the system operates with only nitrogen-containing gas and water as inputs, the presence of these compounds indicates that ammonia must first be produced in situ and reacting further (FIG. 3, element 320). A representative optical image of the stained reaction residue is shown in FIG. 5, visually confirming the presence of amino-functional species.

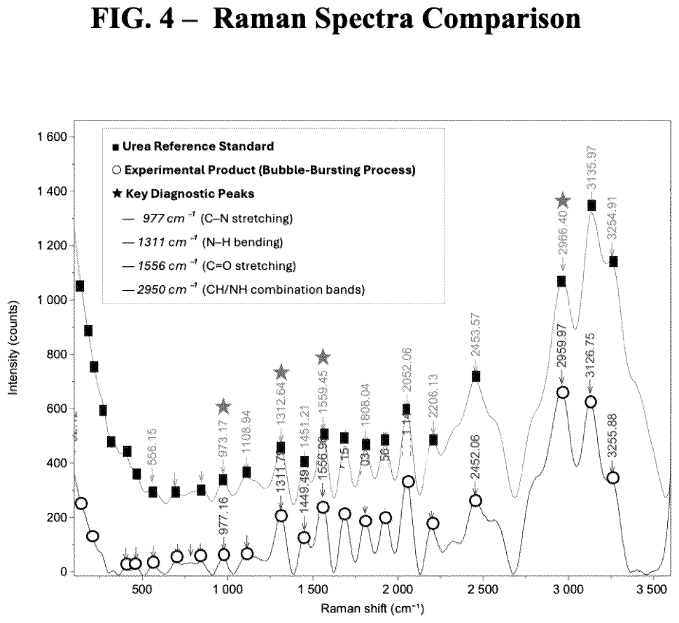

Building on these results, further validation was provided by Raman spectroscopy, which confirmed the presence of urea. The spectrum of the experimental residue showed strong correlation with a commercial urea standard, with coincident peaks at approximately 977 cm⁻¹ (C—N stretch), 1311 cm⁻¹ (N—H bending), and 1556 cm⁻¹ (C═O stretch), consistent with the expected vibrational modes of urea (FIG. 3, element 340; see FIG. 4).

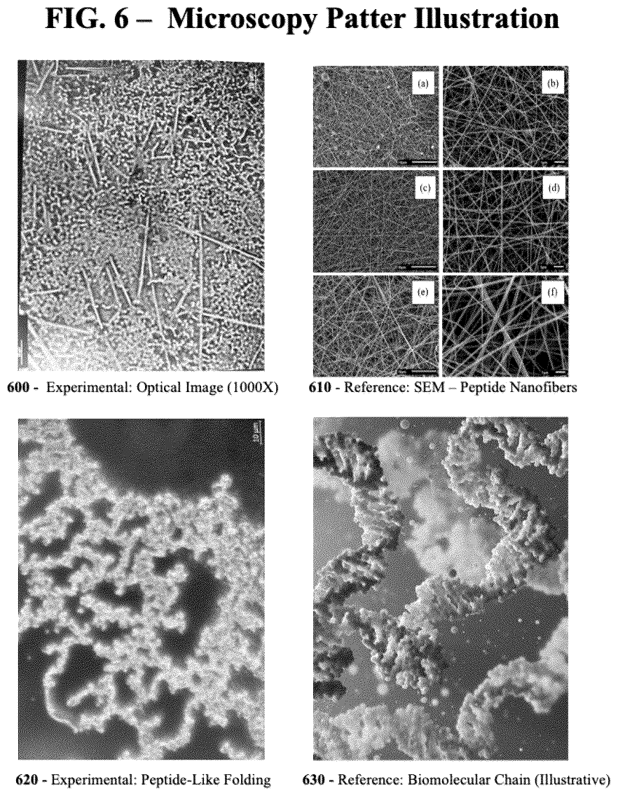

Microscopic analysis further revealed morphological features consistent with peptide-like assemblies. A 1000X optical image of the post-reaction residue (FIG. 6, element 600) showed curdling and aligned structures suggestive of molecular organization, while a dark-field micrograph from a reaction carried out in an aqueous solution containing 30% acetic acid (element 620) displayed pronounced folding patterns indicative of higher-order assembly. For structural context, these results were compared with reference images, including a literature-derived SEM of peptide nanofibers (element 610) and a rendered biomolecular chain model (element 630). The observed similarities in organization and geometry suggest that the bubble-induced environment not only facilitates amino acid formation but also supports spontaneous organization into structured, peptide-like assemblies.

### Reaction Pathways and Variations

Following the observed results, ammonia is understood to act as a key intermediate, reacting with co-solutes to form the nitrogen-based products shown in the following pathways. Such transformations are typically associated with elevated temperatures or specialized conditions in conventional systems. In contrast, they occur here under ambient conditions, driven by localized bubble energy.

In its most fundamental form, the system enables the direct formation of ammonium nitrate from atmospheric air (or nitrogen gas) and water. Under bubble-bursting conditions, ammonia and reactive nitrogen species are generated nearly simultaneously and rapidly combine within the aqueous medium to form ammonium nitrate.

In the presence of dissolved carbon dioxide, ammonia is understood to react to form ammonium carbamate, which subsequently converts to urea (FIG. 3, element 340). The overall transformation —NH₃+CO₂→NH₂COONH₄→CO(NH₂)₂— is well documented in conventional urea synthesis, which typically relies on elevated temperatures and pressures.

When reacted with simple carbon-based acids-such as acetic acid-ammonia yields amino acids, for example, glycine (FIG. 3, element 350). In this pathway, the amine group from ammonia is believed to bond to the carbon adjacent to the carboxyl group in acetic acid, resulting in the formation of glycine (NH₂CH₂COOH), the simplest amino acid.

Alternatively, ammonia may combine with inorganic anions—such as chloride, nitrate, sulfate, or phosphate—to form ammonium salts (FIG. 3, element 360) that serve as nitrogen-based fertilizers. Product selectivity can be further tuned by modifying the chemical composition of the reaction medium or by incorporating additional organic scaffolds to support varied nitrogen-containing product outcomes. This capacity to generate both organic and inorganic products from a single ammonia intermediate underscores the system's versatility and practical relevance across biochemical and agricultural domains.

### System Parameters and Enhancements

In addition to the core reaction conditions, several system parameters may be adjusted to influence product outcomes. The gas feed may include atmospheric air, purified molecular nitrogen (N₂), or mixtures containing trace nitrogen compounds. The system is also compatible with isotopic variants, such as nitrogen-15 (¹⁵N₂), carbon-13 (¹³CO₂), and carbon-14 (¹⁴CO₂), enabling labeled product synthesis for applications in diagnostics, research, and metabolic tracing. These variations do not alter the core process mechanics but may influence downstream product handling, monitoring strategies, or purification requirements.

While designed to operate without external energy input, the system may benefit from optional low-energy enhancements. These include ultraviolet (UV) irradiation, ultrasonic agitation, and high-shear mixing. As illustrated in FIG. 1, such augmentations may be implemented using external components arranged around or within the reaction chamber. These inputs serve to increase the intensity and frequency of bubble rupture and collapse events, enhancing the generation of reactive intermediates and potentially improving reaction yield and product selectivity. For example, ultrasonic transducers may be mounted to the chamber walls to induce controlled cavitation, while UV LEDs may be positioned above the solution to contribute photon energy that assists in bond cleavage or radical formation.

### Applications and Deployment Scenarios

The invention is readily adaptable for both batch and continuous-flow configurations. For continuous operation, the system may be integrated into a modular housing with inlet and outlet ports for fluid circulation. Its design—requiring only a gas source, water, and basic bubble-diffusing hardware—enables deployment across a wide range of operational contexts, including distributed or point-of-use scenarios. Compact embodiments are suitable for small-scale applications, while larger setups can support continuous or high-volume production.

One of the key advantages of the system is its ability to synthesize target compounds directly in a water-based environment, eliminating the need for intermediate storage, transportation, or external infrastructure. This feature enables real-time, on-site chemical conversion—particularly beneficial in environments where decentralized or responsive production is required. Resources can be transformed and utilized immediately, minimizing logistical complexity and enhancing operational flexibility.

In agricultural settings, the system supports efficient on-site generation of nitrogen-based products such as ammonium salts and urea, making it well-suited for both industrial-scale hydroponic operations and smaller-scale installations. In hydroponic farms, the system provides a sustainable and continuous supply of fertilizers tailored to crop-specific nutrient demands, reducing reliance on external inputs. Home gardeners can also benefit from compact implementations, enabling custom fertilizer production with minimal environmental impact.

Beyond agriculture, the invention is applicable in biochemical and pharmaceutical contexts where the synthesis of amino acids and peptides is essential for research and development. The ability to operate under ambient conditions, combined with scalability and minimal equipment requirements, makes the system particularly attractive for laboratory use, small-batch production, and decentralized manufacturing. By enabling synthesis at the point of use, the system reduces overhead and supports agile, resource-efficient workflows.

### DEFINITION OF TERMS

Ambient Conditions: Ambient conditions refer to standard laboratory or environmental conditions, typically around room temperature (20-25° C.) and atmospheric pressure (approximately 1 atm), without the application of external heat, pressure, or high-intensity energy sources.

Ammonia (NH₃): As used herein, ammonia refers to a nitrogen-hydrogen compound generated in situ during the reaction process, functioning as a key intermediate in the formation of downstream nitrogen-based products such as urea, amino acids, and ammonium salts.

Ammonium Salt: An ammonium salt refers to a compound formed by the reaction of ammonia with an inorganic acid, producing NH₄⁺ paired with a counterion such as chloride, sulfate, nitrate, or phosphate. These salts are often used as nitrogen-based fertilizers.

Aqueous Solution: An aqueous solution refers to any liquid-phase reaction medium primarily composed of water, which may include dissolved gases, acids, or other solutes such as carbon dioxide or acetic acid to facilitate specific transformation pathways under ambient conditions.

Atomic Hydrogen: Atomic hydrogen refers to monatomic hydrogen (H), a highly reactive species believed to be generated during bubble-bursting events. It readily participates in bond activation and plays a key role in nitrogen fixation pathways under ambient conditions.

Bubble Burst: Bubble burst refers to the collapse or rupture of a gas bubble within a liquid medium, generating a localized release of energy. These events create transient microenvironments that can initiate molecular dissociation and enable chemical transformations.

Gas-Liquid Interface: The gas-liquid interface refers to the boundary zone between the gas introduced into the system and the surrounding aqueous medium. Microbubble activity at this interface contributes to the generation of localized high-energy environments during the reaction process.

High-Energy Microenvironment: A high-energy microenvironment refers to a localized region within the reaction medium where transient physical conditions—such as pressure, temperature, or reactive energy—temporarily rise due to microbubble activity, enabling chemical transformations that would not normally occur under ambient conditions.

In Situ: In situ refers to chemical reactions or intermediate formations that occur directly within the reaction medium, without requiring the isolation, transfer, or external processing of intermediates such as ammonia prior to downstream transformation.

Isotopic Nitrogen: Isotopic nitrogen refers to nitrogen atoms containing a different number of neutrons than naturally occurring nitrogen-14. For example, nitrogen-15 (¹⁵N) is a stable isotope used for labeling or tracing nitrogen atoms in synthetic and biochemical studies.

Isotopic Carbon: Isotopic carbon refers to carbon atoms containing a different number of neutrons than naturally occurring carbon-12. For example, carbon-13 (¹³C) and carbon-14 (¹⁴C) are stable and radioactive isotopes, respectively, commonly used for tracing, labeling, or dating purposes in chemical and biochemical applications.

One-Step Process: A one-step process refers to a chemical transformation in which the desired product is synthesized directly from starting materials within a single reaction environment, without requiring intermediate purification, catalyst addition, or sequential process steps.

Raman Spectrum: A Raman spectrum is a graphical representation of inelastic light scattering caused by molecular vibrations. Peaks in the spectrum correspond to specific vibrational modes, allowing for compound identification based on characteristic frequency shifts relative to incident light.

Reactive Nitrogen Species: Reactive nitrogen species (RNS) refer to chemically reactive forms of nitrogen, such as atomic nitrogen or nitrogen radicals, generated during bubble-burst events. These species are capable of initiating or participating in bond formation with hydrogen or carbon atoms.

Ruhemann's Purple: Ruhemann's purple is a deep purple chromophore produced when ninhydrin reacts with free amine groups. The color change serves as a well-established indicator for detecting amino-functional compounds, particularly in the context of amino acid synthesis verification.

Urea: Urea (CO(NH₂)₂) refers to a nitrogen-based compound synthesized from ammonia and carbon dioxide. It is commonly used in fertilizers and industrial formulations and is formed in this system under ambient conditions via the intermediate ammonium carbamate pathway.

What is claimed is:

1. A method for synthesizing a nitrogen-containing compound under ambient temperature and pressure conditions and without the use of an external catalyst, the method comprising:
(a) introducing a nitrogen-containing gas into an aqueous solution;
(b) generating microbubbles of the nitrogen-containing gas within the aqueous solution using a submerged bubble-diffusing component; and
(c) inducing expansion and collapse of the microbubbles to generate high-energy microenvironments sufficient to initiate molecular dissociation and form reactive nitrogen species in situ, whereby ammonia is formed as a reactive intermediate.

2. The method of claim 1, wherein the nitrogen-containing gas comprises atmospheric air or molecular nitrogen.

3. The method of claim 1, wherein the aqueous solution contains dissolved carbon dioxide.

4. The method of claim 1, wherein the nitrogen-containing compound is urea, formed by reaction of ammonia with carbon dioxide.

5. The method of claim 1, wherein the nitrogen-containing compound is glycine, formed by reaction of ammonia with acetic acid.

6. The method of claim 1, wherein the nitrogen-containing compound is an ammonium salt formed by reaction of ammonia with an anion selected from the group consisting of chloride, nitrate, sulfate, and phosphate.

7. The method of claim 1, wherein the nitrogen-containing compound is an amino acid formed by reaction of ammonia with a carbon-based acid.

8. The method of claim 1, further comprising one or more energy-augmenting components configured to increase the frequency or intensity of bubble-burst events.

9. The method of claim 1, wherein the nitrogen-containing compound is synthesized on-site for use in hydroponic farming, agriculture, or biochemical production.

10. A system for synthesizing a nitrogen-containing compound under ambient temperature and pressure conditions and without the use of an external catalyst, comprising:
(a) a reaction chamber containing an aqueous solution and at least one co-solute;
(b) a gas supply configured to deliver a nitrogen-containing gas into the aqueous solution; and
(c) a submerged bubble-diffusing component configured to generate microbubbles of the nitrogen-containing gas within the aqueous solution,
wherein collapse or rupture of the microbubbles creates high-energy microenvironments that promote the in situ formation of ammonia, which reacts with the at least one co-solute to produce a nitrogen-containing compound.

11. The system of claim 10, wherein the gas supply comprises atmospheric air or molecular nitrogen.

12. The system of claim 10, further comprising an energy-augmenting component selected from the group consisting of an ultraviolet light source, an ultrasonic transducer, and a high-shear mixing element.

13. A nitrogen-containing compound selected from the group consisting of urea, an amino acid, and an ammonium salt, produced by the method of claim 1.

14. The compound of claim 13, wherein one or more atoms are isotopically enriched or labeled with an isotope selected from the group consisting of nitrogen-15, carbon-13, carbon-14, deuterium, and oxygen-18.

15. The compound of claim 13, wherein the nitrogen-containing compound is configured for use as a fertilizer.
